(19) United States
(12) Patent Application Publication
(10) Pub. No.: US 2015/0301040 A1
(43) Pub. Date: Oct. 22, 2015

(54) IDENTIFICATION OF MODULATORS OF BINDING PROPERTIES OF ANTIBODIES REACTIVE WITH A MEMBER OF THE INSULIN RECEPTOR FAMILY

(71) Applicants: CHARITE - UNIVERSITÄTSMEDIZIN BERLIN, BERLIN (DE); ICI IMMUNOCHEMICAL INTELLIGENCE GMBH, BERLIN (DE)

(72) Inventors: Lutz SCHOMBURG, Berlin (DE); Waldemar MINICH, Berlin (DE); Siegmund KARASCH, Berlin (DE)

(21) Appl. No.: 14/380,294

(22) PCT Filed: Feb. 25, 2013
(86) PCT No.: PCT/EP2013/053707

§ 371 Date: Aug. 21, 2014

(30) Foreign Application Priority Data
Feb. 24, 2012 (EP) 12156930.5

(51) Int. Cl. G01N 33/564

(52) U.S. Cl. CPC G01N 33/564; G01N 2333/912; G01N 2500/02; G01N 2500/20; G01N 2800/042

(57) ABSTRACT

The present invention relates to methods and kits useful in the identification of modulators of the binding properties of antibodies reactive with one or more members of the insulin receptor family selected from the insulin receptor (IR), the insulin-like growth factor 1 receptor (IGF1R), the insulin-like growth factor 2 receptor (IGF2R), the insulin-IGF1 hybrid receptor (IIHR), and the insulin receptor-related receptor (IRRR) and methods for the detection of such antibodies.

The present invention relates to methods and kits useful in the identification of modulators of the binding properties of antibodies reactive with one or more members of the insulin receptor family and for the detection of such antibodies.

### BACKGROUND OF THE INVENTION

The insulin receptor family (IRF) mainly consists of four receptors that bind insulin and/or IGF1 with different affinities: insulin receptor (IR), IGF1 receptor (IGF1R), and IGF2 receptor (IGF2R), and the insulin-IGF1 hybrid receptor (IIHR). Another orphan member of this family is the insulin receptor-related receptor (IRRR).

From the above mentioned receptors, IR, IGF1R, IIHR, and IRRR belong to the family of ligand-activated tyrosine kinase receptors, whereas IGF2R is a transmembrane monomer with a large extracellular domain and no intrinsic signaling capabilities.

IR and IGF1R are expressed at the cell surface as homodimers composed of two identical monomers, or as heterodimers composed of two different receptor monomers (e.g., the insulin-IGF1 hybrid receptor (IIHR)). IIHRs are widely distributed in different mammalian tissues. They behave in a manner similar to IGF1R with respect to ligand-induced tyrosine phosphorylation signaling.

Both IGF1R and IR receptors are composed of two alpha and two beta glycosylated subunits, wherein two transmembrane alpha-beta chains form together a disulfide-linked heterotetramer (beta-alpha-alpha-beta). The receptors have an extracellular ligand binding domain, a single transmembrane domain, and a cytoplasmic domain exhibiting the tyrosine kinase activity. The extracellular domain is composed of the entire alpha subunits and a portion of the N-terminus of the beta subunits, while the intracellular portion of the beta subunits contains the tyrosine kinase (TK) domain.

While similar in structure, IGF1R and IR serve different physiological functions. IR is primarily involved in metabolic functions, whereas IGF1R mediates growth and differentiation. However, their ligands insulin and IGF-1 can induce both mitogenic and metabolic effects when binding the receptors.

Due to the structural relationship of the members of the IRF, the ligand insulin does not only bind to IR, but also to IGF1R and IIHR, the ligand IGF-1 does not only bind to IGF1R, but also to IR, IIHR, and IGF2R, and the ligand IGF-2 does not only bind to IGF2R, but also to IR, IIHR, and IGF1R.

Autoantibodies are known to play an important role in the generation of autoimmune disorders. For example, the presence of autoantibodies to IR in a subject indicates the onset of diabetes (Type B insulin resistance).

Furthermore, non-endogenous antibodies to IGF1R are currently in development to provide novel therapeutical approaches in the treatment of cancer. For example, the drug candidate's figitumumab, developed by Pfizer Inc. and dalatozumab, developed by Merck & Co Inc. are monoclonal therapeutical antibodies to IGF1R, which are currently investigated for the treatment of various types of cancer. For example, patent applications WO 2002/053596 A, WO 2004/083248 A, WO 2005/016967 A, WO 2005/016970 A, US 2005/0249730, US 2005/0084906, WO 2005/052005 A, WO 2005/058967 A, WO 2005/094376 A, WO 2006/008639 A, WO 2006/013472 A, and WO 2008/077546 A refer to the treatment of cancer by means of anti-IGF1R antibodies.

The prevalence and role of autoantibodies which are capable to bind members of the IRF is not yet fully understood and requires further investigation to successfully allow diagnosis, prevention, and treatment of autoimmune disorders (such as, e.g., insulin dependent diabetes mellitus (IDDM), Morbus Basedow, Graves Orbitopathy), obesity, neurological disorders, growth disorders, cancer generation and development (including breast, colon, ovarian, prostate, lung), and other cellular proliferative disorders and other diseases or dysfunctions. For example, dysfunctional IR signaling has been reported in disorders including diabetes type I and II, dementia, and cancer.

More specific and more sensitive assay methods may be helpful to develop a deeper understanding of these complex pathways and will serve as valuable tools in the diagnosis and research in this context, e.g., by allowing discrimination of different physiological states between a healthy individual and progressing dysfunctions. In particular, the measurement of the autoantibodies to IRF members as serological markers can be useful in the early diagnosis or differential diagnosis, in prevention and/or therapy of any of the above mentioned autoimmune disorders.

Furthermore, the improvements of present invention are enabling the identification of modulators (i.e. activators, inhibitors or otherwise affecting or triggering molecules) of the interaction of the members of the IRF and analyte antibodies that affect or trigger the receptor activity, and which therefore may be suitable as pharmaceutically effective compounds in the diagnosis, prevention and/or treatment of any of the diseases which are related to members of the IRF.

Expression of a recombinant human IGF1R-luciferase fusion protein in stably transfected HEK293 cells has been shown by W. Minich et al. (Detection of Autoantibodies to the Insulin-Like Growth Factor-1 Receptor in Patients with Graves Orbitopathy by Luminescent Immunoprecipitation Analysis. Poster Presentation ITC 2010 Paris) who showed quantification of auto-antibodies to IGF1R in sera of patients with Graves' orbitopathy using an immunoprecipitation assay (IPA).

Verch et al. (Bioanalysis September 2011, 3(18): 2107-2117) reported the development of two ELISA assays for the quantification of therapeutical anti-IGF1R antibodies in order to study their pharmacokinetic properties. These two assays are based on classical ELISA methods using labeled anti-human IgG antibodies or labeled anti-human IgGFc antibodies as the detecting reagent.

Furthermore, an ELISA Kit for human anti-insulin receptor antibody detection is provided by Uscn Life Science Inc. (Wuhan, C N). This kit is based on the principle of a sandwich enzyme immunoassay, wherein a microtiter plate has been pre-coated with the antigen. After incubation with the sample, biotin-conjugated antigen is added, and HRP-conjugated avidin is used as the detecting reagent.

Generally, it is known that the use of labeled anti-IgG- or anti-IgGFc-antibodies as the detecting reagent may form complexes with unspecifically antigen-bound antibodies, thereby resulting in false positive signals. Similarly, the biotin-avidin amplification technique is known to result in poor signal to noise ratios. Therefore, there is still the need for assay technology and performance improvement, for example, in the area of high throughput screening (HTS-) assays, where sample size is often critical and both consumption of sample volumes and assay reagents need always further reduction due to limited availabilities and the requirement of cost reduction. Moreover, epidemiological analyses or HTS-assays profit from minimal reaction step numbers, low hands-on-time during assay handling and other technical improvements.

Here, the present invention, overcomes the aforementioned problems by providing improved detection methods and kits employed in the analysis and screening of antibodies reactive with antigenic molecules which are selected from the IRF. In particular, by providing the new immunoassay methods and kits, the present invention surprisingly improves specificity and sensitivity of prior art antibody detection as discussed above. The use of the methods according to the invention is much closer to the physiological conditions and therefore allows the antigenic molecules to expose their correct three-dimensional structure. Consequently, the methods according to the present invention are not only favorable for performing automated assay formats in research and clinical laboratories but are more sensitive and specific than prior art assays.

Surprisingly, it was found by means of the methods according to the present invention that autoantibody-positive samples do cross-react with different members of the IRF. Due to the surprisingly improved sensitivity and specificity of the methods according to the present invention, the new assay format allows for a more differentiated investigation and development of novel therapeutics and diagnostic tools in the prophylaxis and treatment of disorders and diseases related to members of the IRF. Furthermore, it is possible to perform the method according to the invention with any kind of a bivalent antibody suitable according to the present invention regardless from which source the antibody is derived from.

### SUMMARY OF THE INVENTION

Therefore, it is one embodiment of the present invention to provide a method for the identification of a modulator of the binding properties of analyte antibodies reactive with one or more antigenic molecules, said method comprising: (a) providing one or more analyte antibodies reactive with one or more first and one or more second antigenic molecules; (b) providing one or more first antigenic molecules with which said analyte antibodies can interact and which first antigenic molecule is selected from the insulin receptor family (IRF); and (c) providing one or more second antigenic molecules with which analyte antibodies can interact and which second antigenic molecule is selected from the IRF; and (d) contacting said analyte antibodies as provided by step (a) and said first antigenic molecules as provided by step (b) and said second antigenic molecules as provided by step (c) simultaneously or successively with a sample to be investigated, whereby said modulator when present in said sample can interact with the said analyte antibodies and/or said antigenic molecules so as to interfere with the formation of complexes comprising [first antigenic molecule]-[analyte antibody]-[second antigenic molecule]; and (e₁) prior to, or concurrent with, or subsequent to, step (d), providing immobilizing means whereby said first antigenic molecule as present in the said complexes formed in step (d), respectively, as capable to form complexes in step (d) can be immobilized to a solid support prior to, or concurrent with, or subsequent to, step (d); and/or (e₂) prior to, or concurrent with, or subsequent to, step (d), providing second labeling means whereby said first antigenic molecule as present in the said complexes formed in step (d), respectively, as capable to form complexes in step (d) is labeled with said second labeling means prior to, or concurrent with, or subsequent to, step (d); and (f) prior to, or concurrent with, or subsequent to, step (d), providing first labeling means whereby said second antigenic molecule as present in the said complexes formed in step (d), respectively, as capable to form complexes in step (d) is labeled with said first labeling means prior to, or concurrent with, or subsequent to, step (d); and (g) prior to, or concurrent with, or subsequent to, step (d), providing one or more modulators capable to interact with the complexes formed in step (d) and/or capable to interfere with the complex formation according to step (d) and contacting said one or more modulators simultaneously or successively with the said sample, the said one or more first antigenic molecules, and/or the said one or more second antigenic molecules prior to, or concurrent with, or subsequent to step (d), or contacting said one or more modulators simultaneously or successively with the said complexes formed in or subsequent to step (d); and (h) detecting the presence of complexes formed in or subsequent to step (d).

It is another embodiment of the present invention to provide a method of detecting in a sample to be investigated the presence and/or the binding properties of analyte antibodies reactive with one or more antigenic molecules, said method comprising: (a) providing one or more first antigenic molecules with which analyte antibodies when present in said sample can interact and which first antigenic molecule is selected from the insulin receptor family (IRF); and (b) providing one or more second antigenic molecules with which analyte antibodies when present in said sample can interact and which second antigenic molecule is selected from the IRF; and (c) contacting said first antigenic molecules as provided by step (a) and said second antigenic molecules as provided by step (b) simultaneously or successively with the sample to be investigated, whereby analyte antibodies when present in said sample can interact with said antigenic molecules so as to form complexes comprising [first antigenic molecule]-[analyte antibody]-[second antigenic molecule]; and (d₁) prior to, or concurrent with, or subsequent to, step (c), providing immobilizing means whereby said first antigenic molecule as present in the said complexes formed in step (c), respectively, as capable to form complexes in step (c) can be immobilized to a solid support prior to, or concurrent with, or subsequent to, step (c); and/or (d₂) prior to, or concurrent with, or subsequent to, step (c), providing second labeling means whereby said first antigenic molecule as present in the said complexes formed in step (c), respectively, as capable to form complexes in step (c) is labeled with said second labeling means prior to, or concurrent with, or subsequent to, step (c); and (e) prior to, or concurrent with, or subsequent to, step (c), providing first labeling means whereby said second antigenic molecule as present in the said complexes formed in step (c), respectively, as capable to form complexes in step (c) is labeled with said first labeling means prior to, or concurrent with, or subsequent to, step (c); and (g) detecting the presence of complexes formed in or subsequent to step (c) so as to provide indication of analyte antibodies present in said sample. Furthermore, the said method according to the invention can be used for the identification of a modulator of the binding properties of the said analyte antibodies reactive with one or more antigenic molecules in combination with the general knowledge of the person skilled in the art of screening for active compounds with the potential for use as a pharmaceutically active compound.

The methods according to the present invention are, preferably, in vitro methods. Moreover, they may comprise steps in addition to those explicitly mentioned above including sample pretreatments or evaluation of the results (e.g., with respect to the detection of the presence of complexes formed) obtained by the methods. The methods may be carried out manually and/or assisted by automation. Preferably, one or more of steps (a), (b), (c), (d), (d₁), (d₂), (e), (e₁), (e₂), (f) and/or (g) may in total or in part be assisted by automation including suitable robotic and sensory equipment for detection and/or a computer-implemented processing and/or analysis in step (g). For a person skilled in the art, it is known that the methods according to the present invention require calibration or standardization to compare the detected signals, e.g. of the presence of complexes with (one or more) known amounts of reference complex formation.

In other embodiments of the present invention the said first antigenic molecules and the said second antigenic molecules are identical or, alternatively, they are not identical (i.e. different). In other embodiments of the present invention the said first antigenic molecules and the said second antigenic molecules are not identical and belong to different members of the IRF. In other embodiments of the present invention the said first antigenic molecules and/or the said second antigenic molecules are embedded in a membrane environment. In still other embodiments the methods of the present invention allow the detection of an antibody against a member of the IRF of about 3 ng/ml, preferably of about 1 ng/ml, more preferred of about 0.3 ng/ml, even more preferred of about 0.1 ng/ml and most preferred of about 0.03 ng/ml.

In other embodiments of the methods of the present invention the one or more first antigenic molecules, which are immobilized to a solid support, are provided prior to step (c) of the method of detecting autoantibodies, respectively, prior to step (d) of the method for the identification of modulators according to the present invention.

The one or more first antigenic molecules may be immobilized to a solid support prior to contact with a sample to be investigated. Optionally, the solid support may be provided in a liquid phase (e.g., dispersion, suspension, and colloid). Such immobilized one or more first antigenic molecules are subsequently contacted with the said sample either simultaneously or successively with contact of the said sample and with one or more second antigenic molecules. The immobilized one or more first antigenic molecules when contacted with the said sample may form intermediate complexes comprising [first antigenic molecule]-[analyte antibody] wherein the one or more first antigenic molecule is immobilized to a solid support and the thus formed immobilized intermediate complex is subsequently contacted with the one or more second antigenic molecules, present in solution, so as to form the hitherto described complexes comprising [first antigenic molecule]-[analyte antibody]-[second antigenic molecule] directly or indirectly immobilized to a solid support via the first antigenic molecule.

In other embodiments of the method according to the present invention the one or more first antigenic molecules, which are provided with a labeling means, are provided prior to step (c) of the method of detecting autoantibodies, respectively, prior to step (d) of the method for the identification of modulators according to the present invention.

In another embodiment of the methods according to the present invention the one or more second antigenic molecules, which are provided with a labeling means, are provided prior to step (c) of the method of detecting autoantibodies, respectively, prior to step (d) of the method for the identification of modulators according to the present invention.

In another embodiment of the methods according to the present invention the one or more first antigenic molecules, which are immobilized to a solid support and the one or more second antigenic molecules, which are provided with a first labeling means, are both provided prior to step (c) of the method of detecting autoantibodies, respectively, prior to step (d) of the method for the identification of modulators according to the present invention.

In another embodiment of the methods according to the present invention the one or more first antigenic molecules, which are provided with a second labeling means and the one or more second antigenic molecules, which are provided with a first labeling means are both provided prior to step (c) of the method of detecting autoantibodies, respectively, prior to step (d) of the method for the identification of modulators according to the present invention.

In another embodiment of the methods according to the present invention the one or more first antigenic molecules, which are both immobilized to a solid support and provided with a second labeling means and the one or more second antigenic molecules, which are provided with a first labeling means are all provided prior to step (c) of the method of detecting autoantibodies, respectively, prior to step (d) of the method for the identification of modulators according to the present invention.

In still other embodiments of the present invention the one or more first antigenic molecules are immobilized to a solid support and the one or more second antigenic molecules are provided with labeling means, whereby the one or more second antigenic molecules are provided in solution.

In yet other embodiments of the present invention the one or more first antigenic molecules are immobilized to a solid support and the one or more second antigenic molecules are provided with labeling means, whereby the one or more first antigenic molecules and the one or more second antigenic molecules are provided in solution.

Another embodiments of the methods of the present invention comprise directly monitoring the interaction of (i) analyte antibodies present in the sample and (ii) one or more first antigenic molecules and (iii) one or more second antigenic molecules and as provided by the present invention, by employing assay techniques substantially as known in the art (e.g., non-competitive or competitive assays), for example of the sandwich type or RET type, the latter assay type not requiring immobilization and separation of the one or more first antigenic molecules from the liquid phase.

Yet another embodiment of the methods of the present invention allow for the identification of modulators, which are capable to interact with the complexes formed in step (c) and/or which are capable to interfere with the complex formation according to step (c) as defined in the method of detecting analyte antibodies according to the present invention.

Accordingly, another embodiment of the methods of detecting analyte antibodies of the present invention further comprises step (f) prior to, or concurrent with, or subsequent to, step (c), providing one or more modulators capable to interact with the complexes formed in step (c) and/or capable to interfere with the complex formation according to step (c) and contacting said one or more modulators simultaneously or successively with the said sample, the said one or more first antigenic molecules, and/or the said one or more second antigenic molecules prior to, or concurrent with, or subsequent to step (c), or contacting said one or more modulators simultaneously or successively with the said complexes formed in or subsequent to step (c).

In another embodiments of the methods according to the present invention the one or more modulators are provided prior to step (c) of the method of detecting autoantibodies, respectively, prior to step (d) of the method for the identification of modulators according to the present invention.

In still other embodiments of the methods according to the present invention one or more of the said means selected from the group consisting of the said immobilizing means, the said second labeling means, and the said first labeling means are provided prior to step (c) of the method of detecting autoantibodies, respectively, prior to step (d) of the method for the identification of modulators according to the present invention.

In a further embodiment the present invention provides a kit, which is useful for the performance of any of the methods according to the present invention comprising (a) one or more first antigenic molecules selected from the IRF as defined in one or more of the methods according to the invention; (b) one or more second antigenic molecules selected from the IRF as defined in one or more of the methods according to the invention; (c₁) immobilization means as defined in one or more of the methods according to the invention and/or (c₂) second labeling means as defined in one or more of the methods according to the invention; and (d) first labeling means as defined in one or more of the methods according to the invention, and, optionally, one or more analyte antibodies, which are reactive with the one or more first and second antigenic molecules as defined in one or more of the methods according to the invention. In another embodiment the kit according to the present invention comprises (a) said first antigenic molecules labeled with a second labeling means, or (a) said first antigenic molecules immobilized to a solid support; and (b) said second antigenic molecules labeled with a first labeling means.

In still other embodiments the present invention provides the use of any of the methods of the kits according to the invention for the diagnosis of the presence or onset of a disease related to the insulin receptor family and/or for the identification of a pharmaceutically effective compound for the treatment and/or prophylaxis of a disease related to the insulin receptor family.

### DETAILED DESCRIPTION OF THE INVENTION

Principally, the terms and phrases used in this application shall have the meaning of the general knowledge of the person skilled in the art. However, the following preferred meaning of specified terms and phrases may be noted:

The terms “polypeptide” and “protein” are used interchangeable throughout this application.

The term “sample” to be investigated according to the present invention shall preferably mean any sample which is essentially a liquid or suspension, preferably of biological and/or chemical origin. The sample can be obtained by well known techniques and may preferably mean isolated body fluids such as blood, plasma, serum, cerebrospinal fluid, saliva, urine, seminal liquid, tear fluids and others. The sample may further encompass nail clippings, faeces, other excrement, separated cells, cell homogenates, tissue homogenates, or organ homogenates obtained from an animal (e.g., mouse, rat, guinea pig, dog, pig, primates) or human individual. Tissue samples or organ samples may be obtained from any tissue or organ by, e.g., biopsy. Separated cells may be obtained from the body fluids or the tissues or organs by separating techniques such as centrifugation or cell sorting. Preferably, cell samples, tissue samples or organ samples are obtained from those cells, tissues or organs which express, contain, accumulate, concentrate or produce the antigenic molecules referred to herein. The sample may have been subject to treatment and/or modification, known to the person skilled in the art in order to allow storage or further processing in a method of the invention. For example, the person skilled in the art knows that the sample may be diluted in a suitable buffer, or, if the sample is derived from urine, any biotin contained in the sample need to be removed in order to avoid interference with accurate biotin determination, if biotin is used as a label means in the method of the invention. The sample may be selected from an individual suspected of the onset or presence of a dysfunctional condition selected from autoimmune disorders, growth, development or energy metabolism disorders, growth hormone binding protein disorder, growth hormone receptor disorder and growth hormone disorders. In one embodiment of the invention, the sample may comprise a known amount of analyte antibodies and/or one or more modulators, preferably, a known amount of both analyte antibodies and one or more modulator. This may be of particular relevance for the identification of modulators as hereinbefore described.

Generally, the term “antigenic molecule” according to the present invention means any molecule with which an analyte antibody can interact and which is capable of binding an (one or more) analyte antibody to form specific complexes comprising [analyte antibody-antigenic molecule]. The antigenic molecule may be natural or synthetic and modifications thereto are preferably such as to not detrimentally affect the binding properties in the methods according to the present invention.

The term “analyte antibodies” according to the present invention means any antibody capable of binding to any member of the IRF, respectively, capable of binding to one or more of the receptors selected from the group consisting of IR, IGF1R, IGF2R, IIHR, and IRRR as further outlined herein, whose presence is being quantitatively and/or qualitatively analyzed. The term “analyte antibody” may include a monoclonal antibody, a polyclonal antibody, a single chain antibody, a bispecific antibody or diabody, a bivalent antibody, a multispecific antibody, a synthetic antibody, an aptamer, a spiegelmer, a human or humanized antibody, and a fragment or variant thereof such as, e.g., Fab, Fv or scFv fragments, or a chemically modified derivative of any of these, e.g. antibody-drug conjugates, domain antibodies, nanobodies or antibody mimetica (DARPins, designed ankyrin repeat proteins). In specific embodiments of the present invention the term “analyte antibodies” means endogenous autoantibodies, therapeutic antibodies, and/or diagnostic antibodies.

The term “member of the insulin receptor family”, respectively, “insulin receptor family” (IRF) according to the present invention means any polypeptide selected from the group consisting of the insulin receptor (IR), the insulin-like growth factor 1 receptor (IGF1R), the insulin-like growth factor 2 receptor (IGF2R), the insulin-IGF1 hybrid receptor (IIHR), the insulin receptor-related receptor (IRRR), any subunit, variant, analogue, derivative, and fragment thereof. Preferably, the IRF is of animal (e.g., mouse, rat, guinea pig, dog, pig, primates) or human origin, more preferred human.

In one embodiment of the methods of the invention the term “member of the insulin receptor family” or “insulin receptor family” (IRF) means the insulin receptor (IR) or any subunit, or any variant thereof. In another embodiment of the present invention only the said first antigenic molecules are selected from the insulin receptor (IR) or any subunit, or any variant thereof.

In a second embodiment of the methods of the invention the term “member of the insulin receptor family” or “insulin receptor family” (IRF) means the insulin-like growth factor 1 receptor (IGF1R) or any subunit, or any variant thereof. In another embodiment of the present invention only the said first antigenic molecules are selected from the insulin-like growth factor 1 receptor (IGF1R) or any subunit, or any variant thereof.

In a third embodiment of the methods of the invention the term “member of the insulin receptor family” or “insulin receptor family” (IRF) means the insulin-like growth factor 2 receptor (IGF2R) or any variant thereof. In another embodiment of the present invention only the said first antigenic molecules are selected from the insulin-like growth factor 2 receptor (IGF2R) or any variant thereof.

In a fourth embodiment of the methods of the invention the term “member of the insulin receptor family” or “insulin receptor family” (IRF) means the insulin-IGF1 hybrid receptor (IIHR) or any subunit, or any variant thereof. In another embodiment of the present invention only the said first antigenic molecules are selected from the insulin-IGF1 hybrid receptor (IIHR) or any subunit, or any variant thereof.

In a fifth embodiment of the methods of the invention the term “member of the insulin receptor family” or “insulin receptor family” (IRF) means the insulin receptor-related receptor (IRRR) or any subunit, or any variant thereof. In another embodiment of the present invention only the said first antigenic molecules are selected from the insulin receptor-related receptor (IRRR) or any subunit, or any variant thereof.

In a sixth embodiment of the methods of the invention the term “member of the insulin receptor family” or “insulin receptor family” (IRF) means any polypeptide selected from the insulin receptor (IR) or any subunit, or any variant thereof and the insulin-like growth factor 1 receptor (IGF1R) or any subunit, or any variant thereof.

In a seventh embodiment of the methods of the invention the term “member of the insulin receptor family” or “insulin receptor family” (IRF) means any polypeptide selected from the insulin receptor (IR) or any subunit, or any variant thereof and the insulin-like growth factor 2 receptor (IGF2R) or any variant thereof.

In an eighth embodiment of the methods of the invention the term “member of the insulin receptor family” or “insulin receptor family” (IRF) means any polypeptide selected from the insulin receptor (IR) or any subunit, or any variant thereof and the insulin-IGF1 hybrid receptor (IIHR) or any subunit, or any variant thereof.

In a ninth embodiment of the methods of the invention the term “member of the insulin receptor family” or “insulin receptor family” (IRF) means any polypeptide selected from the insulin receptor (IR) or any subunit, or any variant thereof and the insulin receptor-related receptor (IRRR) or any subunit, or any variant thereof.

In a tenth embodiment of the methods of the invention the term “member of the insulin receptor family” or “insulin receptor family” (IRF) means any polypeptide selected from the insulin-like growth factor 1 receptor (IGF1R) or any subunit, or any variant thereof and the insulin-like growth factor 2 receptor (IGF2R) or any variant thereof.

In an eleventh embodiment of the methods of the invention the term “member of the insulin receptor family” or “insulin receptor family” (IRF) means any polypeptide selected from the insulin-like growth factor 1 receptor (IGF1R) or any subunit, or any variant thereof and the insulin-IGF1 hybrid receptor (IIHR) or any subunit, or any variant thereof.

In a twelfth embodiment of the methods of the invention the term “member of the insulin receptor family” or “insulin receptor family” (IRF) means any polypeptide selected from the insulin-like growth factor 1 receptor (IGF1R) or any subunit, or any variant thereof and the insulin receptor-related receptor (IRRR) or any subunit, or any variant thereof.

In a thirteenth embodiment of the methods of the invention the term “member of the insulin receptor family” or “insulin receptor family” (IRF) means any polypeptide selected from the insulin-like growth factor 2 receptor (IGF2R) or any variant thereof and the insulin-IGF1 hybrid receptor (IIHR) or any subunit, or any variant thereof.

In a fourteenth embodiment of the methods of the invention the term “member of the insulin receptor family” or “insulin receptor family” (IRF) means any polypeptide selected from the insulin-like growth factor 2 receptor (IGF2R) or any variant thereof and the insulin receptor-related receptor (IRRR) or any subunit, or any variant thereof.

In a fifteenth embodiment of the methods of the invention the term “member of the insulin receptor family” or “insulin receptor family” (IRF) means any polypeptide selected from the insulin-IGF1 hybrid receptor (IIHR) or any subunit, or any variant thereof and the insulin receptor-related receptor (IRRR) or any subunit, or any variant thereof.

Suitable polypeptides and their corresponding genes, which encode the members of the IRF, are well known to the skilled person in the art. Members of the IRF are commercially available from recombinant sources (e.g. from R&D Systems, Inc., Minneapolis, Minn. 55413, USA, OriGene Technologies, Inc., Rockville, Md. 20850, USA) and are well known from protein and nucleic acid sequence databases, such as, e.g., EMBL, Genbank and others. Currently available database accession numbers for members of the IRF are given for the human species at the specific receptor proteins, however, the present invention shall not be understood to be limited thereto.

The term “insulin receptor” (IR) according to the present invention means any isolated polypeptide having a naturally occurring amino acid sequence or any variant thereof. The amino acid sequences and gene sequences encoding IR are well known to the skilled person, e.g., from entries in sequence databases such as UniProtKB, e.g., P06213 (1382 amino acid length from Homo sapiens). In another embodiment of the present invention the IR is modified for use in in vitro analysis so that it does not comprise a functional intact tyrosine kinase domain or may completely lack the intracellular tyrosine kinase domain due to deletion, e.g., by use of recombinant technologies in protein modification, which are well known to the person skilled in the art. In still another embodiment of the present invention, IR means an IR variant, which exhibits pathogenic or dysfunctional prevalence in an animal (e.g., mouse, rat, guinea pig, dog, pig, primates) or human subject. In yet another embodiment of the present invention the IR is embedded in a membrane environment.

The term “insulin-like growth factor 1 receptor” (IGF1R) according to the present invention means any isolated polypeptide having a naturally occurring amino acid sequence or any variant thereof. The amino acid sequence and gene sequence encoding IGF1R are well known to the skilled person, e.g., from entries in sequence databases such as UniProtKB, e.g., P08069 (1367 amino acid length from Homo sapiens) or C9J5X1 (1366 amino acid length from Homo sapiens). In another embodiment of the present invention the IGF1R is modified for use in in vitro analysis so that it does not comprise a functional intact tyrosine kinase domain or may completely lack the intracellular tyrosine kinase domain due to deletion, e.g., by use of recombinant technologies in protein modification, which are well known to the person skilled in the art. In still another embodiment of the present invention, IGF1R means an IGF1R variant, which exhibits pathogenic or dysfunctional prevalence in an animal (e.g., mouse, rat, guinea pig, dog, pig, primates) or human subject. In yet another embodiment of the present invention the IGF1R is embedded in a membrane environment.

The term “insulin-like growth factor 2 receptor” (IGF2R) according to the present invention means any isolated polypeptide having a naturally occurring amino acid sequence or any variant thereof. The amino acid sequence and gene sequence encoding IGF2R are well known to the skilled person, e.g., from entries in sequence databases such as UniProtKB, e.g., P11717 (2491 amino acid length from Homo sapiens). In another embodiment of the present invention, IGF2R means an IGF2R variant, which exhibits pathogenic or dysfunctional prevalence in an animal (e.g., mouse, rat, guinea pig, dog, pig, primates) or human subject. In still another embodiment of the present invention the IGF2R is embedded in a membrane environment.

The term “insulin-IGF-1 hybrid receptor” (IIHR) according to the present invention means any isolated polypeptide having a naturally occurring amino acid sequence or any variant thereof. The amino acid sequence and gene sequence encoding IIHR are well known to the skilled person. In another embodiment of the present invention the IIHR is modified for use in in vitro analysis so that it does not comprise a functional intact tyrosine kinase domain or may completely lack the intracellular tyrosine kinase domain due to deletion, e.g., by use of recombinant technologies in protein modification, which are well known to the person skilled in the art. In still another embodiment of the present invention, IIHR means an IIHR variant, which exhibits pathogenic or dysfunctional prevalence in an animal (e.g., mouse, rat, guinea pig, dog, pig, primates) or human subject. In yet another embodiment of the present invention the IIHR is embedded in a membrane environment.

The term “insulin-receptor related receptor” (IRRR) according to the present invention means any isolated polypeptide having a naturally occurring amino acid sequence or any variant thereof. The amino acid sequence and gene sequence encoding IRRR are well known to the skilled person, e.g., from entries in sequence databases such as UniProtKB, e.g., P14616 (1297 amino acid length from Homo sapiens). In another embodiment of the present invention the IRRR is modified for use in in vitro analysis so that it does not comprise a functional intact tyrosine kinase domain or may completely lack the intracellular tyrosine kinase domain due to deletion, e.g., by use of recombinant technologies in protein modification, which are well known to the person skilled in the art. In still another embodiment of the present invention, IRRR means an IRRR variant, which exhibits pathogenic or dysfunctional prevalence in an animal (e.g., mouse, rat, guinea pig, dog, pig, primates) or human subject. In yet another embodiment of the present invention the IRRR is embedded in a membrane environment.

The term “variant” according to the present invention means any fragment, analog, derivative, fusion protein, subunit or subunit chain of a molecule mentioned. Preferably, the variant may have at least essentially the same biological properties as the respective polypeptides or proteins mentioned, except for the tyrosine kinase activity, immobilizing features and/or labeling features. Moreover, it is to be understood that the term “variant” according to the present invention shall include an amino acid sequence which differs due to at least one amino acid substitution, modification, deletion and/or addition, wherein the amino acid sequence of the variant is still, preferably, at least about 50%, at least about 60%, at least about 70%, at least about 80%, at least about 85%, at least about 90%, at least about 92%, at least about 95%, at least about 97%, at least about 98%, or at least about 99% identical with the amino sequence of the respective polypeptide or protein mentioned, preferably over the entire length of the specific polypeptide or protein. Variants may be allelic variants or any other species specific homologs, paralogs, or orthologs. Moreover, the variants referred to herein include fragments of the respective polypeptides or proteins mentioned hereinbefore from the IRF, provided these fragments have essentially the same biological properties. Furthermore, the variants referred to herein include fusion proteins of the respective polypeptides or proteins mentioned hereinbefore from the IRF with polypeptides, which are suitable as immobilization means, labeling means, or label, provided such fusion protein essentially maintains the same biological properties of the respective polypeptide or protein mentioned hereinbefore from the IRF. Variants may further include modifications of the said polypeptides or proteins by glycosylation or any other chemical or enzymatic modification, provided these variants have essentially the same biological properties as referred to above.

The variants according to the invention may include so-called ‘silent’ substitutions, additions are deletions which do not alter or substantially alter the biological activity. More particularly, the variants according to the present invention may have been modified with respect to one or more of the amino acid residues, which are substituted by a conserved or non-conserved amino acid residue, preferably a conserved amino acid residue, or such ones in which one or more of the amino acid resides may include a substituted radical. Such variants are deemed to be within the scope of the teachings herein. Most typically, variants are those that vary by conservative amino acid substitutions. Such substitutions are those that substitute a given amino acid in a polypeptide by another amino acid of similar chemical characteristics. In this regard are understood as conservative substitutions the replacements, one for another, among the small aliphatic amino acids A, V, L and I; among the hydroxyl residues S and T; among the acidic residues D and E; among the amide residues N and Q; among the basic residues K and R; and among the aromatic residues F and Y (according to the single letter code of amino acids of the IUPAC nomenclature).

In one embodiment of the present invention, a “variant” shares essentially the same biological properties. In another embodiment of the present invention a “variant” of a member of the IRF is used, which exhibits pathogenic or dysfunctional prevalence, i.e., which exhibits a disease related to the insulin receptor family in an animal (e.g., mouse, rat, guinea pig, dog, pig, primates) or human subject.

According to another embodiment of the present invention the one or more members of the IRF are modified so that they do not comprise a functional intact tyrosine kinase domain or completely lack the intracellular tyrosine kinase domain, e.g., by means of recombinant technologies in protein modification, which are well known to the person skilled in the art.

The term “biological properties” in accordance with the understanding of the present invention means the binding properties of the respective molecule mentioned. In case of a specific member of the IRF it may be its ability to form a complex with insulin, IGF-1, and/or IGF-2 under suitable conditions. For the purpose of the present invention, it is understood that the signaling property of any member of the IRF with respect to the tyrosine kinase activity is expressly excluded. Optionally, the term “biological properties” may further include certain specific immunological properties of polypeptides, e.g., if they are specifically detectable by the same ELISA method. Particularly, a member of the IRF exhibits essentially the same “biological properties” like a variant thereof, if both (a) can interact with the analyte antibodies, (b) are detectable by the same ELISA method and/or (c) are detectable by the detection method according to the present invention.

In another preferred embodiment of the invention the one or more first antigenic molecules and the one or more second antigenic molecules are one or more polypeptides selected from the group consisting of IR, IGF1R, IGF2R, IIHR, and IRRR.

The term “providing prior to step (c) of the method of detecting autoantibodies”, respectively, “providing prior to step (d) of the method for the identification of modulators” according to the present invention shall mean prior to contacting the said antigenic molecules and the said analyte antibodies in a method of the present invention.

The term “detecting the presence and/or the binding properties” according to the present invention means determining the amount or concentration, preferably quantitatively. The measuring can be done directly or indirectly. Direct measuring relates to measuring the amount or concentration of one or more of the reaction educts and/or the reaction products based on a signal which is obtained from the one or more reaction educts and/or the reaction products itself/themselves and the intensity of which directly correlates with the number of molecules of the one or more reaction educts and/or the reaction products in the reaction volume. Such a signal may be obtained, e.g., by measuring the intensity or value of a specific physical or chemical property of the one or more reaction educts and/or reaction products. Indirect measuring includes measuring of a signal obtained from a secondary component (i.e. a component not being the reaction educt or reaction product itself) or a biological read out system, referred to as “label means” in this specification, e.g., of measurable cellular or transmembrane responses, ligands, or enzymatic reaction products, e.g. by means of fluorophors, chromophors, ion concentrations, which suitably is performed by means of optical, electrical and/or electronical equipment. For measurement of enzymatic reaction products, preferably the amount of substrate is saturating. Optionally, the substrate may also be labeled with a detectable label prior to the reaction. Preferably, the reaction partners are contacted with the substrate for an adequate period of time, which corresponds to the time necessary for a detectable amount of the one or more reaction products to be produced such as a measurable signal. Instead of measuring the amount or concentration of the one or more reaction products, the time necessary for appearance of a given (e.g. detectable) amount or concentration of the one or more reaction products can be measured.

According to the present invention, “detecting the presence and/or the binding properties” can be achieved by all means for determining the amount of a reaction educt and/or reaction product known to the skilled person. Said means comprise immunoassay devices and methods which may utilize labeled molecules in various sandwich, competition, or other assay formats. Said assays will develop a signal which is indicative for the presence or absence of the reaction educts and/or the reaction products. Moreover, the signal strength can, preferably, be correlated directly or indirectly (e.g. reverse-proportional) to the amount of reaction educts and/or the reaction products in the reaction volume. Said methods comprise, preferably, biosensors, optical devices coupled to immunoassays, biochips, analytical devices such as spectrometers or chromatography devices. Further, methods include ELISA-based methods using, optionally pre-treated or pre-coated, micro-plates, micro-arrays, or tube-arrays, fully-automated or robotic immunoassays (available, e.g., on Roche-Elecsys™, Abbott-AxSYM™ or Brahms Kryptor™ analyzer systems). Preferably, “detecting the presence and/or the binding properties” comprises the steps which will allow bringing the reaction partners together for an adequate period of time.

The term “binding” according to the present invention includes both covalent and non-covalent binding. The term “specific binding” means a binding affinity of at least 3 times higher, preferably of at least about 10 times higher and more preferably of at least about 50 times higher than the binding affinity to other molecules. In another embodiment of the present invention the term “binding” shall mean the binding of binding partners in an in vitro binding assay under suitable conditions, preferably under conditions according to the assay manufacturer's instructions or according to the methods essentially as defined hereinafter in the Example section. Generally, “binding” means a binding affinity (K_{D}—which means the quotient of dissociation constant to association constant) of about 10⁻¹⁴ M to 10⁻⁷ M, preferably of about 10⁻¹³ M to 10⁻⁹ M.

A “modulator” according to the present invention includes any biological or chemical compound, a macromolecule (e.g. larger than about 5 kDa), a small molecule (e.g., smaller than about 5 kDa or even smaller than about 800 Da), an isolated compound or a mixture of compounds, an extract or homogenates of tissue or organ origin, or a compound of synthetic origin comprising, e.g., one or more peptides, polypeptides (e.g., polyclonal or monoclonal antibodies, including single chain antibodies, diabodies, multispecific antibodies, humanized antibodies, hybrid antibodies, or fragments thereof, such as Fv, Fab and F(ab)₂ fragments), aptamers, spiegelmers, nucleic acids, and/or small molecules.

The term “antigenic molecule embedded in a membrane environment” according to the present invention means that the said antigenic molecule—which is in case of the IRF a transmembrane protein—is provided in a model membrane or the like, which includes any suitable synthetic, natural or artificial environment of a, e.g., cellular, membrane, vesicular, micellar or liposomal structure, whereby the said antigenic molecule is able to interact with the aforementioned analyte antibodies and thus complex formation.

Assay methods for proteins embedded in a membrane environment are well known to the skilled person, e.g., from technologies and assays which use artificial or biomimetical membranes or lipid bilayers (herein “model membranes”). Model membranes are widely used for investigating membrane proteins and many of them are suitable for the performance of the methods of the present invention, particularly, if denaturation of the said antigenic molecule can be avoided.

The term “model membrane” according to the present invention encompasses, e.g., any liposome or vesicle, such as artificially prepared vesicles comprising one, two or more lipid layers in spherical geometry. Such liposomes and vesicles are used as vehicles for the transport of lipids, proteins and small molecules; therefore they are used in the administration of pharmaceuticals. Vesicles or liposomes can easily be prepared by disrupting biological membranes from cells cultures, tissues, organs, or subcellular structures (e.g. nucleus, Golgi, endoplasmatic reticulum, and mitochondria), e.g., by sonication and/or extrusion) and subsequent self-reassembling of the lipid structures, whereby labeling with dyes or labeled polypeptides suitable in RET is easily achievable. Further “model membranes” may comprise lipid bilayers which have been synthetically assembled in vitro. They can be made from one or more synthetic and/or natural lipids.

The term “model membranes” according to the present invention may further include, e.g., black lipid membranes (BLM), vesicles, lipid bilayers, liposomes, micelles, bicelles, hybrid bilayers (e.g. comprising a hydrophobic monolayer and a lipid monolayer), and nanodiscs, which may be anchored, supported or tethered to a solid phase or solid substrate, and which may, optionally, be provided with a spacer or cushion (e.g., polyethylene glycols, oligonucleotides, peptides, polypeptides (e.g., streptavidin), hydrogels and others) which may allow to maintain a distance of the membrane and/or the aforementioned antigenic molecule to the solid substrate. Preferably, the spacer or cushion is a hydrophilic molecule. In contrast to a vesicle or a cell membrane, the aforementioned supported bilayer may have a planar structure sitting on a solid support. Therefore, only the upper face of the bilayer is exposed to the solution. For example, the preparation of proteoliposomes is well known from European patent application EP 1992688 A1, of which the liposome preparation methods as disclosed in any of examples 1 to 20 are herewith incorporated by reference.

The term “micelles” according to the present invention means another type of model membranes which lack a lipid bilayer. In aqueous solutions, micelles are assemblies of amphipathic molecules (e.g., detergents) with their hydrophilic heads exposed to solvent and their hydrophobic tails in the center. Micelles are able to solubilize membrane proteins by partially encapsulating them and shielding their hydrophobic surfaces from solvent. The term “bicelles” means still another type of model membranes which are typically made of two lipids, one of which forms a lipid bilayer while the other forms an amphipathic, micelle-like assembly shielding the bilayer center from surround solvent molecules. The term “nanodiscs” means a segment of a bilayer encapsulated by an amphipathic protein coat, a lipid or detergent layer. Membrane proteins can be incorporated into and solubilized by nanodiscs.

The term “disease related to the insulin receptor family” according to the present invention preferably means any dysfunction which is related to one or more polypeptides selected from the group consisting of IR, IGF1R, IGF2R, IIHR, and IRRR, more preferably of one, two, or three selected polypeptides of the said IRF. The term “disease related to the insulin receptor family” according to the present invention encompasses autoimmune disorders (such as, e.g., insulin dependent diabetes mellitus (IDDM), Morbus Basedow, Graves Orbitopathy) and also disorders and dysfunctions such as obesity, neurological disorders, growth disorders, cancer generation and development (including breast, colon, ovarian, prostate, lung), and other cellular proliferative disorders and dysfunctions, whether of autoimmune origin or not, preferably of autoimmune origin.

The term “immobilizing means” according to the present invention means any reagent and/or process, which is suitable to immobilize the said first antigenic molecules to a solid support according to the knowledge of the person skilled in the art. With respect to the kind of a solid support and conditions employed in accordance with the present invention, the solid support and conditions do generally not fundamentally differ from conventionally used solid supports and conditions employed in known immunoassay techniques. A solid support for use according to the present invention can comprise an ELISA plate as currently employed in known ELISA techniques, or may employ any other suitable support for use in the present invention, such as micro-titer plates (having 96, 384, 1536, or 3456 wells or more) or parts thereof, tubes, particles, magnetic beads, nitrocellulose or the like. Materials suitable as solid support which can be used in accordance with the teachings of the present invention are well known in the art and include, e.g., commercially available column materials, polystyrene beads and other carriers, latex beads, magnetic beads, colloidal metal, glass surfaces and chips for use in protein microchip technologies, silanylated surfaces and chips for use in protein microchip technologies, silicon surfaces and chips for use in protein microchip technologies, nitrocellulose carriers, cellulose carriers, membranes, model membranes, stabilized liposomes or cells (e.g., duracytes), wells and walls of reaction trays or microtiter plates, plastic tubes etc.

The antigenic molecules as hereinbefore defined may be immobilized to any carrier which is known to the person skilled in the art. Examples of such carriers include glass, polystyrene, polyvinyl chloride, polypropylene, polyethylene, polycarbonate, dextran, nylon, amylose, natural and modified cellulose, polyacrylamide, agarose, magnetite, and gold. The carrier can be either soluble or insoluble, in case of an insoluble carrier; the carrier is a solid or colloid and may, optionally, be provided as suspension.

Suitable methods for immobilizing said antigenic molecules are well known and include, but are not limited to ionic, hydrophobic, covalent interactions and the like. It may also be suitable to use solid immobilization means in suspension according to the present invention, wherein the carrier is provided in suspension, e.g. a microbead or microsphere consisting of different microbeads or microspheres, optionally labeled, both carrying each different molecules. Methods of producing such suspensions, e.g., based on solid-phase chemistry and photo-labile protective groups, are, e.g., known from U.S. Pat. No. 5,744,305.

The term “labeling” according to the present invention means labeling by direct or indirect methods. Direct labeling involves coupling of the label directly (covalently or non-covalently) to the molecule to be labeled. Indirect labeling involves binding (covalently or non-covalently) of a second ligand to the molecule to be labeled. Such second ligand should specifically bind to the molecule to be labeled with an at least 3-fold higher, preferably at least 10-fold, and more preferred at least 50-fold higher affinity under assay conditions. Said second ligand may be coupled with a suitable label means and/or may bind a third ligand binding to the second ligand. The use of second, third, or even higher order ligands is often used to increase the signal. Suitable second and higher order ligands may include antibodies, secondary antibodies, and the well-known streptavidin-biotin system (Vector Laboratories, Inc.). Furthermore, the molecule to be labeled or the substrate may also be “tagged” with one or more tags known in the art. Such tags may then be targets for higher order ligands. Suitable tags include biotin, digoxygenin, His-Tag, Glutathion-S-Transferase, FLAG-tag (N-DYKDDDDK-C), green fluorescence protein (GFP), myc-tag, influenza a virus haemagglutinin (HA), maltose binding protein, and others. In the case of a peptide or polypeptide, the tag is generally located at or close to the N-terminus and/or C-terminus.

Furthermore, the molecule to be labeled or the substrate may also be provided with a suitable “spacer” known in the art in order to avoid in case of bulky molecules any limitations with respect to the binding properties due to spatial constrictions.

The term “first labeling means” according to the invention shall preferably mean any direct or indirect detectable labeling means selected from the group of enzymatic labels, isotopic or radioactive labels, chemoluminescent labels, bioluminescent labels, fluorescent labels, magnetic labels (e.g. “magnetic beads”, including paramagnetic and superparamagnetic labels), dye labels (chromophors), and others known in the art. Suitable labels are detectable by an appropriate detection method known in the art. Suitable labels may further include gold particles, latex beads, acridan ester, luminol, and ruthenium. Preferably suitable are non-radioactive labels.

Enzymatically active labels include, e.g., horseradish peroxidase, alkaline phosphatase, beta-galactosidase, luciferase, and derivatives thereof. Suitable substrates for detection include di-amino-benzidine (DAB), 3,3′-5,5′-tetramethyl-benzidine, NBT-BCIP (4-nitro blue tetrazolium chloride and 5-bromo-4-chloro-3-indolyl-phosphate, CDP-Star™ (Amersham Biosciences), ECL Biosciences) and others known in the art.

A suitable enzyme-substrate combination may result in increase or decrease of a colored reaction product (chromophor), fluorescence, or chemo- or bioluminescence, which can be measured according to methods known in the art (e.g. using a photometer, a photo-multiplier, and a light-sensitive film or camera system). The same principles apply for measuring the endpoint or performance or development of an enzymatic reaction.

Suitable fluorescence labels include fluorescent dyes and proteins (such as GFP and its derivatives), Cy3, Cy5, Texas Red, fluorescein, and the Alexa dyes (e.g. Alexa 568). Further suitable fluorescent labels are commercially available e.g. from Molecular Probes (Oregon, USA). Also the use of quantum dots as fluorescent labels is encompassed. Examples of fluorescent proteins include, but are not limited to, green, yellow, cyan, blue, and red fluorescent proteins.

Suitable chemoluminescence or bioluminescence labels include, but are not limited to prokaryotic (e.g., bacterial lux-encoded) or eukaryotic (e.g., firefly luc-encoded) luciferases, as well as variants possessing varied or altered optical properties, such as luciferases that produce different colors of light, e.g. derived from Photinus pyralis, from the sponge Suberities domuncula, and the Mycena fungi. Furthermore, photoproteins, e.g., calcium-activated photoproteins and their specifically designed variants may be suitable, which are capable of producing light typically in the range of 200 nm to 1100 nm, or in the visible spectrum (i.e., between approximately 350 nm and 800 nm), e.g., obelin from the marine polyp Obelia longissima, or Aequorin, e.g., from the luminescent jellyfish Aequorea victoria or from other organisms may be suitable, optionally in a membrane.

Suitable radioactive labels include ³⁵S, ¹²⁵I, ³²P, ³³P and the like. A radioactive label can be detected by any method known and appropriate, e.g. a light-sensitive film or a phosphor imager.

Suitable detection methods according to the present invention also include precipitation (particularly immunoprecipitation), electrochemiluminescence (electrically generated chemiluminescence), biolominescence, RIA (radioimmunoas say), ELISA (enzyme-linked immunosorbent assay), sandwich enzyme immune tests, sandwich immunoassays (ECLIA), dissociation-enhanced lanthanide fluorescent Immunoassay (DELFIA, PerkinElmer Inc., USA), scintillation proximity assay (SPA), turbidimetry, nephelometry, latex-enhanced turbidimetry or nephelometry, latex agglutination assay, or solid phase immune assays.

Further methods known in the art (such as gel electrophoresis, 2D gel electrophoresis, SDS polyacrylamid gel electrophoresis (SDS-PAGE), Western Blotting, and mass spectrometry), can optionally be used in combination with the labeling or other detection methods as described above.

The one or more antigenic molecules according to the invention may be directly or indirectly provided with the labeling means, substantially as hereinbefore described. The term “second labeling means” according to the invention shall preferably mean any direct or indirect detectable labeling means selected from the group of chemoluminescent labels, bioluminescent labels, fluorescent labels, dye labels (chromophors), and others known in the art. The use of the term “second labeling means” means that the “second labeling means” is not identical with the “first labeling means”. Suitable second labeling means are detectable by an appropriate detection method known in the art based on resonance energy transfer (RET) principle. Preferably, “second labeling means” include labeling means suitable for fluorescence resonance energy transfer (FRET), bioluminescence resonance energy transfer (BRET), or chemoluminescence resonance energy transfer (CRET) as known to the person skilled in the art. The selection of a suitable second labeling means will consider the kind of the first labeling means in order to safeguard that the RET signal is detectable and are known to the person skilled in the art. Furthermore, the RET signal generation and selection of suitable labeling means provides the person skilled in the art with additional information about the kind and kinetics of complex formation and the structural features of the complexes formed.

### Sequences:

Seq. ID No. 1 Primer P1 (33mer)
Seq. ID No. 2 Primer P2 (34mer)
Seq. ID No. 3 Primer P3 (35mer)
Seq. ID No. 4 Primer P4 (33mer)
Seq. ID No. 5 DNA encoding amino acids 2-551 from firefly luciferase
Seq. ID No. 6 amino acid sequence 2-551 from firefly luciferase
Seq. ID No. 7 DNA encoding amino acids 1-1367 from human IGF1R
Seq. ID No. 8 amino acid sequence 1-1367 from human IGF1R
Seq. ID No. 9 DNA encoding amino acids 1-1919 from human IGF1R-luc fusion
Seq. ID No. 10 amino acid sequence 1-1919 from human IGF1R-luc fusion
Seq. ID No. 11 DNA encoding amino acids 1-1370 from human insulin receptor
Seq. ID No. 12 amino acid sequence 1-1370 from human insulin receptor (IR)
Seq. ID No. 13 DNA encoding amino acids 1-1922 from human IR-luc fusion
Seq. ID No. 14 amino acid sequence 1-1922 from human insulin receptor-luc fusion
Seq. ID No. 15 DNA encoding amino acids 1-927 from the extracellular domain of human IGF (ECDhIGF1R) Seq. ID No. 16 amino acid sequence 1-927 from the extracellular domain of human
IGF1R (ECDhIGF1R)
Seq. ID No. 17 Primer P5 (33mer)
Seq. ID No. 18 Primer P6 (33mer)
Seq. ID No. 19 Primer P7 (36mer)
Seq. ID No. 20 Primer P8 (83mer)

The present invention will now be illustrated by the following examples, which shall be understood not to limit the scope of the present invention in any way.

### Experimental Procedures

### Materials

DNA primers (P1-P8) were obtained from BioTeZ Berlin Buch GmbH (Berlin, Germany); pSP-luc+NF vector was obtained from Promega GmbH (Mannheim, Germany); pIRESneo vector was obtained from Clontech (Palo Alto, Calif., USA); vector pCR-XL-TOPO-IR was obtained from ImaGenes GmbH (Berlin, Germany); vector pFastBac1 was obtained from Invitrogen; Goat anti human IgG (SIGMA) was labeled with Acridinium NHS Ester (Cayman Chemical Company, Ann Arbor, Mich., USA); IGF1 was obtained from Acris Antibodies (Herford, Germany); Insulin was obtained from Sigma-Aldrich; anti-IGF1R (Tyr1165/Tyr1166) antibody was obtained from Novus Biologicals (Edinburgh, UK); CellTiter-Glo luminescent cell viability assay kit was obtained from Promega GmbH; High Five insect cells were purchased from Invitrogen; Polystyrene tubes coated with PGA14 antibody (Selenotest LIA) were obtained from ICI immunochemical intelligence GmbH (Berlin, Germany). If not otherwise stated, all other reagents and chemicals were obtained from Sigma-Aldrich Chemie GmbH (Munich, Germany) or Merck KGaA (Darmstadt, Germany); enzymes were obtained from Promega or New England Biolabs (Ipswich, Mass., USA).

### Example 1

### Construction of Fusion Proteins

### Example 1A

### Construction of an IGF1R-Luciferase Fusion Protein

The DNA (Seq. ID No. 5) encoding amino acids 2-551 from firefly luciferase (Seq. ID No. 6 on pSP-luc+NF) was amplified by PCR using primers P1 (Seq. ID No. 1) and P2 (Seq. ID No. 2) containing EcoRI and BamHI restriction sites, respectively.

pIRESneo was digested with EcoRI and BamHI restriction endonucleases; the obtained fragment was replaced with the DNA encoding firefly luciferase obtained from the aforementioned PCR resulting in plasmid pIRESneo-Luc.

The DNA (Seq. ID No. 7) encoding amino acids 1-1367 from human IGF1R (Seq. ID No. 8) was amplified by PCR using primers P3 (Seq. ID No. 3) and P4 (Seq. ID No. 4) containing NotI and EcoRI restriction sites, respectively. pIRESneo-Luc was digested with NotI and BamHI restriction endonucleases and the obtained fragment was replaced with the DNA sequence encoding human IGF1R obtained from the aforementioned PCR resulting in vector pIRESneo-IGF1R-Luc containing Seq. ID No. 9 encoding Seq. ID No. 10.

### Example 1B

### Construction of an IR-Luciferase Fusion Protein

The DNA (Seq. ID No. 11 of pCR-XL-TOPO-IR) encoding amino acids 1-1370 from human IR (Seq. ID No. 12) was amplified by PCR using primers P5 (Seq. ID No. 17) and P6 (Seq. ID No. 18) containing HpaI and MfeI restriction sites, respectively. pIRESneo-Luc was digested with EcoRV and EcoRI restriction endonucleases and the obtained fragment was replaced with the DNA sequence encoding human IR obtained from the aforementioned PCR resulting in vector pIRESneo-IR-Luc containing Seq. ID No. 13 encoding Seq. ID No. 14.

### Example 1C

### Construction of a Fusion Protein of the Extracellular Domain of IGF1R (ECDhIGF1R) and the 16 Amino Acid Epitope Recognized by PGA14 Antibodies

The DNA (Seq. ID No. 15) encoding 927 amino acids of the extracellular domain of the human IGF1 receptor (Seq. ID No. 16, ECDhIGF1R) was amplified by PCR using primers P7 (Seq. ID No. 19) and P8 (Seq. ID No. 20) containing BamHI and HindIII restriction sites, respectively (P8 containing the coding sequence for the 16 amino acid epitope recognized by PGA14 antibodies). pFastBac1 vector was digested with BamHI and HindIII restriction endonucleases and the obtained fragment was replaced with the DNA sequence encoding the ECD of hIGF1R obtained from the aforementioned PCR resulting in vector pFastBac1-IGF1R-PGA14tag.

### Example 2

### Generation of IGF1R-Luc and IR-Luc Fusion Protein Producing Cells

### Example 2A

### Generation of IGF1R-Luc and IR-Luc Producing HEK 293

HEK 293 cells were grown in DMEM supplemented with 10% fetal bovine serum. Cells were cultivated in a 5% CO2 atmosphere at 37° C. HEK 293 cells were transfected with pIRESneo-IGF1R-Luc vector or pIRESneo-IR-Luc vector using FuGENE6 transfection reagent (obtained from Roche Deutschland Holding GmbH, Grenzach-Wyhlen, Germany) according to the manufacturer's instruction. 48 hours after transfection, selection was started with 0.8 mg/ml G418 (Gibco™ BRL, Invitrogen). Stable clones expressing high levels of fusion protein were selected.

### Example 2B

### Generation of Recombinant Baculovirus Expressing ECDhIGF1R-PGA14tag Fusion Protein

The ECDhIGF1R-PGA14tag sequence obtained from example 1C was transferred to bacmid DNA by site-specific recombination in bacteria. The bacmid was then used to generate a fully recombinant baculovirus in Sf9 insect cells according to the protocols supplied by the manufacturer (Bac-to-Bac expression system manual, Invitrogen).

### Example 2C

### Production of IGF1R-PGA14tag Fusion Protein

Suspension High Five insect cells were grown in Express Five serum-free medium to density 2×10e6 cells/ml. Cells were then infected with recombinant IGF1R-PGA14tag-baculovirus at a multiplicity of infection (MOI) of 1. 72 hours post-infection cell medium was collected and stored at −80 C.°.

### Example 3

### Control of Expression Products

### Example 3A

### Interaction of IGF1R-Luc Cells with IGF1

Confluent HEK 293 cells grown in a 96 well plate were washed with DMEM/F12 medium and incubated with acridinium labeled IGF1 (about 1.0e06 RLU/well) diluted in 100 μl of DMEM/F12, 0.1% BSA for 3 h at 37° C. in a 5% CO₂ atmosphere. In further experiments unlabelled IGF1 (0.3 mg/ml) was added together with acridinium labeled IGF1 to determine unspecific binding. After incubation cells were washed with DMEM, resuspended in PBS containing 2% triton X-100 and obtained lysates were measured in a luminometer Berthold Technologie AutoLumat Plus LB 953 for 10 sec. Each value represents the mean value (RLU)+/−standard deviation (SD) of duplicate measurements (relative light units (RLU)×1000).

TABLE 1

| | Wildtype HEK 293 | IGF1R-Luc HEK 293 |
|---|---|---|
| Labeled IGF1 | 27 +/− 4 | 55 +/− 7 |
| Labeled IGF1 + | 11 +/− 1 | 12 +/− 1 |
| excess IGF1 | | |

This result indicates that IGF1R-luc fusion proteins are correctly processed and expressed at the cell membrane.

### Example 3B

### Interaction of IR-Luc Cells with Insulin

Confluent HEK 293 cells grown in a 96 well plate were washed with DMEM/F12 medium and incubated with acridinium labeled insulin about 1.0e06 RLU/well) diluted in 100 μl of DMEM/F12, 0.1% BSA for 3 h at 37° C. in a 5% CO₂ atmosphere. In further experiments unlabelled insulin (0.3 mg/ml) was added together with acridinium labeled insulin to determine unspecific binding. After incubation the cells were washed with DMEM, resuspended in PBS containing 2% triton X-100 and obtained lysates were measured in a luminometer Berthold Technologie AutoLumat Plus LB 953 for 10 sec. Each value represents the mean value (RLU)+/−standard deviation (SD) of duplicate measurements (RLU×1000).

TABLE 2

| | Wild type HEK 293 | IR-Luc HEK 293 |
|---|---|---|
| Labeled insulin | 32 +/− 2 | 78 +/− 6 |
| Labeled insulin + | 13 +/− 2 | 17 +/− 3 |
| excess insulin | | |

This result indicates that IR-luc fusion proteins are correctly processed and expressed at the cell membrane.

### Example 4

### Preparation of IGF1R-Luc and IR-Luc Cell Extract

Confluent HEK 293 cells (producing either IGF1R-Luc or IR-Luc) grown in a 75 cm² plate were resuspended by scraping into PBS and were washed in the same buffer by centrifugation at 2500 rpm. The resulting cells were lysed in 0.5 ml buffer containing 20 mM HEPES-NaOH pH 7.5, 50 mM NaCl, 1% Triton X-100, 10% glycerol. The suspension was centrifuged at 5,000 rpm for 15 min and the supernatant was collected and stored at −80 C.

### Example 5

### Immunoprecipitation Assay for IGF1R Autoantibodies

The IGF1R-Luc cell extract was diluted 10 times with buffer containing 20 mM HEPES-NaOH pH 7.5, 50 mM NaCl, 1% Triton X-100, 10% glycerol, 5 mg/ml BSA. For immunoprecipitation, 100 μl of diluted extract (about 1.0e07 RLU) was mixed with 10 μl of a sample (serum probe) and incubated overnight at 4° C. Immune complexes were precipitated by addition of 100 μl of 10% protein A-sepharose suspension in same buffer for 1 h at room temperature with shaking. Protein A-sepharose was pelleted and washed 3 times with 1 ml of washing buffer (10 mM Tris-HCl, pH 7.5, 60 mM NaCl, 0.02% Tween 20). Finally, luciferase activity was measured in a Berthold luminometer (AutoLumat Plus LB 953) for 10 sec. Results were expressed as RLU bound. Each value represents the mean value of duplicate measurements. The ability of crude sera and isolated IgG preparations from the same sera IgG to immunoprecipitate the IGF1 receptor was compared.

TABLE 3

| | Serum | RLU-serum | IgG | RLU-IgG |
|---|---|---|---|---|
| | [RLU] | [% of max] | [RLU] | [% of max] |
| 1 | 417 | 0 | 238 | 0 |
| 2 | 619 | 0 | 181 | 0 |
| 3 | 781 | 1 | 392 | 1 |
| 4 | 669 | 0 | 414 | 1 |
| 5 | 348 | 0 | 293 | 0 |
| 6 | 121585 | 83 | 70472 | 92 |
| 7 | 145972 | 100 | 76178 | 100 |
| 8 | 130683 | 90 | 62441 | 82 |
| 9 | 55840 | 38 | 23002 | 30 |
| 10 | 131560 | 90 | 49699 | 65 |

### Table 4: Dilution, Recovery and Stability of the IGF1R Autoantibody Assay.

(A) Five different sera containing autoantibodies were diluted with incubation buffer and measured in assay as described in example 5. (B) Five sera with IGF1R autoantibodies, five sera without IGF1R autoantibodies and their mixtures (1:1) were analyzed in assay as described in example 5. (C) Mix of five sera was incubated at room temperature (RT) and 4° C. for given periods of time and analyzed in IGF1R autoantibodies assay as described in example 5.

### Table 4A: Dilution Experiments for the IGF1R Autoantibodies Assay.

Five different sera containing autoantibodies were diluted with incubation buffer and measured in assay as described in example 5. Bound luciferase activity was measured as previously described. Each value represents the mean value of duplicate measurements (RLU×1000).

TABLE 4A

| Sera (%) | Serum 1 | Serum 2 | Serum 3 | Serum 4 | Serum 5 |
|---|---|---|---|---|---|
| 100 | 76.3 | 93.7 | 71.8 | 41.3 | 86.6 |
| 50 | 50.2 | 54.6 | 39.6 | 21.5 | 51.8 |
| 25 | 27.1 | 30.1 | 24.2 | 13.9 | 27.6 |
| 10 | 11.6 | 13.9 | 11.4 | 6.6 | 12.2 |

### Table 4B: Recovery Experiments for the IGF1R Autoantibodies Assay.

Five sera without IGF1R autoantibodies (A), five sera with IGF1R autoantibodies (B) and their mixtures (1:1) were analyzed in the assay as described in example 5. Bound luciferase activity was measured as previously described. Each value represents the mean value of duplicate measurements (RLU×1000).

TABLE 4B

| | 1 | 2 | 3 | 4 | 5 |
|---|---|---|---|---|---|
| Serum A | 4.9 | 5.6 | 3.3 | 3.2 | 3.4 |
| Serum B | 63.3 | 76.1 | 60.3 | 25.4 | 63.2 |
| Mix A + B | 35.0 | 42.1 | 33.4 | 15.0 | 37.4 |

### Table 4C: Stability Experiments for the IGF1R Autoantibodies Assay.

Serum with IGF1R autoantibodies was incubated for different time at 4° C. and at room temperature (RT). Serum samples were analyzed in assay as described in example 5. Bound luciferase activity was measured as previously described. Each value represents the mean value of duplicate measurements (RLU×1000).

TABLE 4C

| | 0 days | 5 days | 10 days |
|---|---|---|---|
| 4° C. | 53.0 | 52.5 | 49.4 |
| RT | 53.0 | 52.2 | 53.8 |

### Example 6

### Isolation of Human IgG

1 ml of human serum was mixed with 1 ml PBS and 0.2 ml of protein G-sepharose and incubated overnight at 4° C. by shaking. Protein G-sepharose was pelleted and washed ten times with PBS. Bound IgG were eluted with 25 mM citric acid, pH was adjusted to 7 using 1M Hepes-NaOH, pH 8. Eluted IgG were concentrated to 100 μl using Speedvac at room temperature. IgG was transferred into DMEM/F12 medium using speed filtration on sephadex G25 column.

### Example 7

### Effect of Autoantibodies on IGF1R Autophosphorylation

HepG2 cells were seeded in 96 well plates (10 000 cells/well), incubated 24 h in complete DMEM/F12 medium followed by overnight incubation in serum free DMEM/F12 medium containing 0.1% BSA. After serum starvation cells were incubated with human IgG (10-20 mg/ml) in DMEM/F12 medium for 15 min up to 1 h. After this in some cases IGF1 (1 ng/ml) was added and cells were incubated for the additional 15 min. Cells were washed with PBS containing phosphatase inhibitors and lysed in buffer 20 mM HEPES-NaOH pH 7.5, 50 mM NaCl, 2% triton X100, 10% glycerol, phosphatase inhibitors. Cell lysates were subjected to electrophoresis in 10% SDS PAGE and blotted to nitrocellulose membranes. Tyrosine phosphorylated IGF1R was detected on the blot using an anti-IGF1R (Tyr1165/Tyr1166) antibody. Bands were visualized using enhanced chemiluminescence western blotting detection kit (Amersham ECL Plus, GE Healthcare, General Electric Deutschland Holding GmbH, Frankfurt, Germany).

### Example 8

### Effect of Autoantibodies on Cell Growth

MCF7 cells were seeded at 2500 cells per well (96 well plates) and incubated overnight in complete DMEM/F12 medium. Cells were then starved in serum free DMEM/F12 medium containing 0.1% BSA for 5 h followed by addition of IgG (about 10 mg/ml) from five sera with low (1-5) and five sera with high (6-10) concentrations of autoantibodies against IGF1R (isolated from human patients), 1% FCS and 1 ng/ml IGF1. Cells were incubated for 5 days. The CellTiter-Glo luminescent cell viability kit was used to assess the number of viable cells in culture based on quantification of the ATP present, according to the manufacturer manual. Inhibiting effect of autoantibodies was expressed as Inhibition Index calculated as InI %=100×[(1−(RLU test IgG)/(RLU negative IgG pool)]. Each value represents the mean value of duplicate measurements.

TABLE 5

| Serum | Inhibition Index (%) |
|---|---|
| 1 | −7 |
| 2 | 1 |
| 3 | −3 |
| 4 | −3 |
| 5 | −2 |
| 6 | 20 |
| 7 | 18 |
| 8 | 22 |
| 9 | 16 |
| 10 | 21 |

### Example 9

### Bridge Assays

### Example 9A

### Bridge Assay for the Detection of Autoantibodies Interacting Both with IGF1 Receptor and Insulin Receptor

Polystyrene tubes coated with PGA14 antibody were incubated overnight at 4° C. with 200 μl of SF6 insect cell medium containing IGF1R-PGA14tag. After IGF1R-PGA14tag immobilization tubes were washed twice with 1 ml of buffer 20 mM Tris-HCl pH 7.5, 50 mM NaCl, 10% glycerol. Then each tube was incubated overnight at 4 C.° with a mixture of 100 μl of the same buffer containing 10 mg/ml BSA and 100 μl of a sample (serum probe). Tubes were washed twice with 1 ml of the same buffer and incubated overnight at 4 C.° with 200 μl of IGF1R-Luc (or IR-Luc respectively) diluted in the same buffer with BSA (about 40× 10e6 RLU of luciferase activity). After incubation tubes were washed four times with 1 ml of 20 mM Tris-HCl pH 7.5, 100 mM NaCl, 0.1% triton X100. Finally, luciferase activity was measured in a Berthold luminometer (AutoLumat Plus LB 953) for 10 sec. Results were expressed as RLU (relative light units) bound. Each value represents the mean value of duplicate measurements.

TABLE 6

| | IGF1R-Luc | IGF1R-Luc | IR-Luc | IR-Luc |
|---|---|---|---|---|
| Bridge Assay | [RLU] | [% of max] | [RLU] | [% of max] |
| without serum | 2135 | 5 | 947 | 5 |
| control serum | 5348 | 13 | 2616 | 15 |
| serum 406 | 42240 | 100 | 4814 | 27 |
| serum 522 | 39341 | 93 | 17961 | 100 |
| serum 531 | 36646 | 87 | 12839 | 71 |

### Example 9B

### Detection Limits of Patient Serum

An IGF1R-Ab positive serum was diluted with 20 mM Tris-HCl pH 7.5, 50 mM NaCl, 10% glycerol, 10 mg/ml BSA. The assay was performed as described in Example 9A. Negative serum is defined to give a signal like buffer control.

TABLE 7

| Serum dilution | IGF1R-Luc |
|---|---|
| (v/v) | [RLU] |
| Undiluted | 64651 |
| ¼ | 38829 |
| 1/16 | 20269 |
| 1/64 | 8135 |
| 1/256 | 5383 |
| background | 5369 |
| (negative serum) | |

### Example 9C

### Detection Limits of Commercially Available Anti-IGF1R Antibodies

Monoclonal antibody to the extracellular domain of IGF1R (anti-IGF1R clone#24-75, Millipore, USA) was diluted with 20 mM Tris-HCl pH 7.5, 50 mM NaCl, 10% glycerol, 10 mg/ml BSA. The assay was performed as described in Example 9A.

TABLE 8

| Antibody concentration | IGF1R-Luc |
|---|---|
| (ng/ml) | [RLU] |
| 50000 | 805261 |
| 12500 | 767157 |
| 3125 | 626879 |
| 781 | 475770 |
| 195 | 234773 |
| 49 | 88894 |
| 12 | 27013 |
| 3 | 11831 |
| 0.8 | 8856 |
| 0.2 | 5904 |
| 0.05 | 5369 |
| background | 5418 |

This result indicates that the detection limit of the assay according to the invention is about 0.2 ng/ml antibody concentration.

### Example 10

### Clinical Relevance of the Autoantibody Level Against IRF Member Protein in Humans

Examination of serum samples of 1001 probands (healthy adult humans) were analysed for the presence of insulin receptor autoantibody levels. A prevalence of 10% positive individuals (n=103) was determined comparing autoantibody positive and negative individuals revealing a significant difference in basal glucose concentrations (p=0.03).

1-15. (canceled)

16. A method for identification of a modulator of binding properties of analyte antibodies reactive with one or more antigenic molecules, said method comprising:
(a) providing one or more analyte antibodies reactive with one or more first and one or more second antigenic molecules; and
(b) providing one or more first antigenic molecules with which said analyte antibodies can interact, wherein the first antigenic molecule is selected from the insulin receptor family (IRF); and
(c) providing one or more second antigenic molecules with which analyte antibodies can interact, wherein the second antigenic molecule is selected from the IRF; and
(d) contacting said analyte antibodies as provided by step (a) and said first antigenic molecules as provided by step (b) and said second antigenic molecules as provided by step (c) simultaneously or successively with a sample to be investigated, wherein said modulator when present in said sample can interact with said analyte antibodies and/or said antigenic molecules so as to interfere with the formation of complexes comprising:
[first antigenic molecule]-[analyte antibody]-[second antigenic molecule]; and
(e1)) prior to, or concurrent with, or subsequent to, step (d), providing immobilizing means whereby said first antigenic molecule as present in said complexes formed in step (d) capable of forming complexes in step (d) can be immobilized to a solid support prior to, or concurrent with, or subsequent to, step (d); and/or
(e2) prior to, or concurrent with, or subsequent to, step (d), providing second labeling means, wherein said first antigenic molecule as present in the said complexes formed in step (d) capable of forming complexes in step (d) is labeled with said second labeling means prior to, or concurrent with, or subsequent to, step (d); and
(f) prior to, or concurrent with, or subsequent to, step (d), providing first labeling means, wherein said second antigenic molecule as present in the said complexes formed in step (d) capable of forming complexes in step (d) is labeled with said first labeling means prior to, or concurrent with, or subsequent to, step (d); and
(g) prior to, or concurrent with, or subsequent to, step (d), providing one or more modulators capable of interacting with the complexes formed in step (d) and/or capable of interfering with the complex formation according to step (d) and contacting said one or more modulators simultaneously or successively with the said sample, said one or more first antigenic molecules, and/or said one or more second antigenic molecules prior to, or concurrent with, or subsequent to step (d), or contacting said one or more modulators simultaneously or successively with said complexes formed in or subsequent to step (d); and
(h) detecting the presence of complexes formed in or subsequent to step (d).

17. A method of detecting in a sample to be investigated a presence and/or binding properties of analyte antibodies reactive with one or more antigenic molecules, said method comprising:
(a) providing one or more first antigenic molecules with which analyte antibodies when present in said sample can interact, wherein the first antigenic molecule is selected from the insulin receptor family (IRF); and
(b) providing one or more second antigenic molecules with which analyte antibodies when present in said sample can interact, wherein the second antigenic molecule is selected from the IRF; and
(c) contacting said first antigenic molecules as provided by step (a) and said second antigenic molecules as provided by step (b) simultaneously or successively with the sample to be investigated, wherein analyte antibodies when present in said sample can interact with said antigenic molecules so as to form complexes comprising:
[first antigenic molecule]-[analyte antibody]-[second antigenic molecule]; and
(d1) prior to, or concurrent with, or subsequent to, step (c), providing immobilizing means, wherein said first antigenic molecule as present in said complexes formed in step (c) capable of forming complexes in step (c) can be immobilized to a solid support prior to, or concurrent with, or subsequent to, step (c); and/or
(d2) prior to, or concurrent with, or subsequent to, step (c), providing second labeling means, wherein said first antigenic molecule as present in said complexes formed in step (c) capable of forming complexes in step (c) is labeled with said second labeling means prior to, or concurrent with, or subsequent to, step (c); and
(e) prior to, or concurrent with, or subsequent to, step (c), providing first labeling means, wherein said second antigenic molecule as present in said complexes formed in step (c) capable of forming complexes in step (c) is labeled with said first labeling means prior to, or concurrent with, or subsequent to, step (c); and
(g) detecting the presence of complexes formed in or subsequent to step (c), wherein the detecting provides indication of analyte antibodies present in said sample.

18. The method of claim 17, further comprising:
(f) prior to, or concurrent with, or subsequent to, step (c), providing one or more modulators capable of interacting with the complexes formed in step (c) and/or capable of interfering with the complex formation according to step (c) and contacting said one or more modulators simultaneously or successively with said sample, said one or more first antigenic molecules, and/or said one or more second antigenic molecules prior to, or concurrent with, or subsequent to step (c), or contacting said one or more modulators simultaneously or successively with said complexes formed in or subsequent to step (c).

19. The method of claim 16, wherein said first antigenic molecules and said second antigenic molecules are identical.

20. The method of claim 17, wherein said first antigenic molecules and said second antigenic molecules are identical.

21. The method of claim 16, wherein said first antigenic molecules and/or said second antigenic molecules are embedded in a membrane environment.

22. The method of claim 17, wherein said first antigenic molecules and/or said second antigenic molecules are embedded in a membrane environment.

23. The method of claim 16, wherein said one or more first antigenic molecules and/or said one or more second antigenic molecules are lacking a functionally intact tyrosine kinase domain.

24. The method of claim 17, wherein said one or more first antigenic molecules and/or said one or more second antigenic molecules are lacking a functionally intact tyrosine kinase domain.

25. The method of claim 16, wherein the said analyte antibody to be detected in said sample is an endogenous autoantibody or a monoclonal antibody.

26. The method of claim 17, wherein the said analyte antibody to be detected in said sample is an endogenous autoantibody or a monoclonal antibody.

27. A kit for detection of an antibody, which binds to one or more antigenic molecules selected from the insulin receptor family or for the detection of a modulator of said antibody, comprising:
(a) one or more first antigenic molecules selected from the insulin receptor family of claim 16;
(b) one or more second antigenic molecules selected from the insulin receptor family of claim 16;
(c1) immobilization means of claim 16 and/or
(c2) second labeling means of claim 16; and
(d) first labeling means of claim 16.

28. The kit of claim 27, wherein
(a) said first antigenic molecules are labeled with a second labeling means; and
(b) said second antigenic molecules are labeled with a first labeling means.

29. The kit of claim 27, wherein
(a) said first antigenic molecules are immobilized to a solid support; and
(b) said second antigenic molecules are labeled with a first labeling means.

30. The method of claim 16, further comprising diagnosing presence or onset of a disease related to the insulin receptor family.

30. The method of claim 17, further comprising diagnosing presence or onset of a disease related to the insulin receptor family.

32. The method of claim 16, further comprising identifying a pharmaceutically effective compound for the treatment and/or prophylaxis of a disease related to the insulin receptor family.

33. The method of claim 17, further comprising identifying a pharmaceutically effective compound for the treatment and/or prophylaxis of a disease related to the insulin receptor family.

34. The kit of claim 27 for diagnosing presence or onset of a disease related to the insulin receptor family.

35. The kit of claim 27 for identifying a pharmaceutically effective compound for treatment and/or prophylaxis of a disease related to the insulin receptor family.


SEQUENCE LISTING (20 sequences)

SEQ ID NO: 1 - DNA, 33 bp, Artificial Sequence
  source 1..33: /organism=“Artificial Sequence” /note=“primer P1” /mol_type=“unassigned DNA”
gcggaattcgtcaccgacgccaaaaacataaag

SEQ ID NO: 2 - DNA, 34 bp, Artificial Sequence
  source 1..34: /organism=“Artificial Sequence” /note=“Primer P2” /mol_type=“unassigned DNA”
acgggattcttacacggcgatctttccgcccttc

SEQ ID NO: 3 - DNA, 35 bp, Artificial Sequence
  source 1..35: /organism=“Artificial Sequence” /note=“Primer P3” /mol_type=“unassigned DNA”
acggcggccgcatgaagtctggctccggaggaggg

SEQ ID NO: 4 - DNA, 33 bp, Artificial Sequence
  source 1..33: /organism=“Artificial Sequence” /note=“Primer P4” /mol_type=“unassigned DNA”
acggaattcgcaggtcgaagactggggcagcgg

SEQ ID NO: 5 - DNA, 1653 bp, Photinus pyralis
  source 1..1653: /organism=“Photinus pyralis” /note=“cds” /mol_type=“unassigned DNA”
gtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 6 - protein, 550 aa, Photinus pyralis
VTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 7 - DNA, 4104 bp, Homo sapiens
  source 1..4104: /organism=“Homo sapiens” /note=“IGF1R” /mol_type=“unassigned DNA”
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 8 - protein, 1367 aa, Homo sapiens
  IGF1R
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 9 - DNA, 5760 bp, Artificial Sequence
  source 1..5760: /organism=“Artificial Sequence” /note=“hIGF1R-luc fusion” /mol_type=“unassigned DNA”
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgcgaattcgtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 10 - protein, 1919 aa, Artificial Sequence
  hIGF1R-luc fusion protein
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTCEFVTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 11 - DNA, 4113 bp, Homo sapiens
  source 1..4113: /organism=“Homo sapiens” /note=“cDNA insulin receptor” /mol_type=“unassigned DNA”
atgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcatgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaa

SEQ ID NO: 12 - protein, 1370 aa, Homo sapiens [sequence omitted]

SEQ ID NO: 13 - DNA, 5769 bp, Artificial Sequence
  source 1..5769: /organism=“Artificial Sequence” /note=“human insulin receptor-luc fusion” /mol_type=“unassigned DNA”
atgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcatgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcccaattggtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 14 - protein, 1922 aa, Artificial Sequence
  hIR-luc fusion protein
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPSQLVTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 15 - DNA, 2781 bp, Artificial Sequence
  source 1..2781: /organism=“Artificial Sequence” /note=“extracellular domain of human IGF1R” /mol_type=“unassigned DNA”
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaa

SEQ ID NO: 16 - protein, 927 aa, Artificial Sequence
  extracellular domain of human IGF1R
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAK

SEQ ID NO: 17 - DNA, 33 bp, Artificial Sequence
  source 1..33: /organism=“Artificial Sequence” /note=“Primer P5” /mol_type=“unassigned DNA”
acggttaacatgggcaccgggggccggcggggg

SEQ ID NO: 18 - DNA, 33 bp, Artificial Sequence
  source 1..33: /organism=“Artificial Sequence” /note=“Primer P6” /mol_type=“unassigned DNA”
acgcaattgcagcaagatcttgtcaaaagatgt

SEQ ID NO: 19 - DNA, 36 bp, Artificial Sequence
  source 1..36: /organism=“Artificial Sequence” /note=“Primer P7” /mol_type=“unassigned DNA”
atcggatccaccatgaagtctggctccggaggaggg

SEQ ID NO: 20 - DNA, 83 bp, Artificial Sequence
  source 1..83: /organism=“Artificial Sequence” /note=“Primer P8” /mol_type=“unassigned DNA”
atcaagcttcactttcctgctcctggttgctgattctctgaaagctcactgctgccaagtttggcctggacatagaagaacac